(19) United States
(12) Patent Application Publication
(10) Pub. No.: US 2007/0025937 A1
(43) Pub. Date: Feb. 1, 2007

(54) Cosmetic compositions containing hydroquinone

(75) Inventors: Hani Fares, Somerset, NJ (US); Ruby Jain, Wayside, NJ (US); Sidney Peter Foltis, Nutley, NJ (US); Isabelle Hansenne, Westfield, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'Oreal S.A., Paris (FR)

(21) Appl. No.: 11/192,476

(22) Filed: Jul. 29, 2005

(51) Int. Cl. A61K 8/34

(52) U.S. Cl. 424/62

(57) ABSTRACT

Disclosed are compositions for application to skin, comprising hydroquinone or a derivative thereof, and a cosmetically acceptable vehicle comprising an effective amount of absolute ethanol or an effective amount of an ethanol composition comprising ethanol and methyl alcohol or isopropyl alcohol as a denaturant. Also disclosed are methods of whitening skin and reducing discoloration of hydroquinone compositions.

### BACKGROUND OF THE INVENTION

Generally human skin color is determined hereditarily according to the concentration and distribution of melanin in the skin, but it can also be influenced by environmental or physiological conditions such as solar ultraviolet rays, fatigue, and stress.

It is common for people with colored or even dark skin to wish to lighten the skin, and, with this aim, to use cosmetic or dermatological compositions containing bleaching agents. Compositions comprising bleaching agents are also used to treat skin of individuals that displays dyschromias. These dyschromias are of diverse origin: age (age marks), exposure to UV radiation, pregnancy mask, skin pathology, etc. People with freckles may wish such pigmented spots to be less pronounced. Thus, skin lightening is an important skin care need.

It is known that inhibition of tyrosinase may likely lead to skin lightening via inhibition of melanogenesis. See King, R. A. and C. G. Summers, Dermatologic Clinics, Vol. 6 pp. 217-227 (1988). Tyrosinase is present within the melanosomes in epidermal melanocytes and catalyzes the committed step in the formation of melanin from tyrosine. See Goldsmith, L. A., Physiology, Biochemistry, and Molecular Biology of the Skin, Oxford University Press, pp. 873-903 (N.Y. 1991). Tyrosinase catalyzes the hydroxylation of tyrosine (as a tyrosine hydroxylase) and the oxidation of DOPA to DOPAquinone (as DOPA oxidase). Binding of an inhibitor to the active site of tyrosinase results in decreased melanin formation. See generally Prota, G., Melanins and Melanogenesis, Academic Press, Inc., (San Diego 1992). While the process in which melanin is formed is known, the mechanism which induces melanin synthesis, or the step before the tyrosinase acts, is not yet understood in detail. The substances most commonly used as bleaching agents have tyrosinase inhibiting activities such as hydroquinone and its derivatives.

Any active in any composition, especially when used for topical application (whether for pharmaceutical or cosmetic purposes) must be efficacious, bioavailable, stable when exposed to light, air or to the skin. Should the product be unstable, the breakdown products of the active must be innocuous. Several tyrosinase inhibitors are in the marketplace, including hydroquinone, kojic acid and arbutin. However, there are reports of disadvantages associated with each of these products. For example, kojic acid and arbutin are not very bioavailable and thus of marginal efficacy. As reported in U.S. Pat. No. 6,537,527, hydroquinone is oxidized by air, light and tyrosinase itself. These oxidized products of hydroquinone have been implicated in skin irritation (and perhaps cytotoxicity) and in pigmentation rebound (i.e., initial lightening followed by darkening).

U.S. Pat. No. 2,376,884 teaches inclusion of laevo-ascorbic or cevitamic acid in hydroquinone creams reduces oxidation and discoloration of the hydroquinone. U.S. Pat. No. 4,136,166 teaches hydroquinone compositions that also contain a sunscreen agent. U.S. Pat. No. 6,759,033 B2 teaches cosmetic compositions containing a skin-whitening agent such as hydroquinone and sodium magnesium silicate in an amount to slow decomposition of the composition.

### SUMMARY OF THE INVENTION

A first aspect of the present invention is directed to a composition for application to skin, comprising hydroquinone or a derivative thereof, and a cosmetically acceptable vehicle comprising an effective amount of absolute ethanol or an effective amount of an ethanol composition comprising ethanol and methyl alcohol or isopropyl alcohol as a denaturant.

A second aspect of the present invention is directed to a method of whitening skin, comprising applying to skin a composition comprising hydroquinone or a derivative thereof, and a cosmetically acceptable vehicle comprising an effective amount of absolute ethanol or an effective amount of an ethanol composition comprising ethanol and methyl alcohol or isopropyl alcohol as a denaturant.

A third aspect of the present invention is directed to a method for reducing discoloration of compositions containing hydroquinone, comprising preparing a composition comprising hydroquinone or a derivative thereof, and a cosmetically acceptable vehicle comprising an effective amount of absolute ethanol or an effective amount of an ethanol composition comprising ethanol and methyl alcohol or isopropyl alcohol as a denaturant.

According to the teachings of U.S. Pat. No. 5,968,535, ethyl alcohol that is not intended for foods and confectionery, such as, for example, ethyl alcohol for the preparation of cosmetics, has for tax reasons to be rendered unusable for ingestion by the addition of denaturants. Different denaturants are used for this purpose within Europe and outside Europe. A denaturant that is used frequently throughout the world is diethyl phthalate. In many countries, natural ethereal oils and various individual components, such as, for example, isopropanol, phenylethyl alcohol, musk ketone etc., are also used for denaturing. All known denaturants have disadvantages (some of them considerable) which lower the product quality. Diethyl phthalate, for example, in aerosol formulations causes sneezing and, as is the case for all one-component additives, has an uncontrollable influence on scent development of perfumed additives. Other denaturants, such as, for example, isopropanol and musk ketone, influence the odour character of perfumed products and some of them (musk ketone) are known to be color-unstable.

Applicants have discovered that unlike various ethanol denaturants, use of methyl alcohol or isopropyl alcohol as a denaturant, or ethanol without any denaturant (i.e., absolute ethanol) results in substantially less discoloration or color change of hydroquinone over time.

### BRIEF DESCRIPTION OF THE DRAWINGS

FIGS. 1A-E are photographs of compositions containing 1% hydroquinone and different grades of ethanol after storage at 45° C. for two weeks.

### DETAILED DESCRIPTION OF THE INVENTION

Besides hydroquinone, derivatives of hydroquinone that are susceptible to discoloration that may be due to oxidation or decomposition may also be used. Derivatives include, but are not limited to arbutin, 4-hydroxyanisol, mequinol, HMBE, monobenzone, hydroquinone monomethyl ether, hydroquinone monoethyl ether, t-butyl hydroquinone, 4-[(tetrahydro-2H-pyran-2-yl)oxy]phenol, and 4-[(tetrahydro-2H-thiopyran-2-yl)oxy]phenol. Other derivatives are described in WO 9523780. Further, glucosides of hydroquinone may be useful. These include hexose glucosides such as hydroquinone α-D-glucose, hydroquinone β-D-glucose, hydroquinone α-L-glucose, hydroquinone β-L-glucose, hydroquinone α-D-galactose, hydroquinone β-D-galactose, hydroquinone α-L-galactose, and hydroquinone β-L-galactose; pentose glucoside such as hydroquinone α-D-ribose, hydroquinone β-D-ribose, hydroquinone α-L-ribose, hydroquinone β-L-ribose, hydroquinone α-D-arabinose, hydroquinone β-D-arabinose, hydroquinone α-L-arabinose; and hydroquinone β-L-arabinose: amino acid glucosides such as hydroquinone α-D-glucosamine, hydroquinone β-D-glucosamine, hydroquinone α-L-glucosamine, hydroquinone β-L-glucosamine, hydroquinone α-D-galactosamine, hydroquinone β-D-galactosamine, hydroquinone α-L-galactosamine, and hydroquinione β-L-galactosamine; uronic acid glucosides such as hydroquinone α-D-glucuronic acid, hydroquinone β-D-glucuronic acid, hydroquinone α-L-glucuronic acid, hydroquinone β-L-glucuronic acid, hydroquinone α-D-galacturonic acid, hydroquinone β-D-galacturonic acid, hydroquinone α-L-galacturonic acid, and hydroquinone β-L-galacturonic acid.

The amount of hydroquinone or derivative generally ranges from about 0.1% to about 10%, and preferably from about 0.5% to about 5.0%, based on total weight of the cosmetic composition.

The compositions of the present invention also comprise a cosmetically acceptable vehicle that may act as a diluent, dispersant or carrier for the hydroquinone and the other materials that may be present in the composition, so as to facilitate their distribution when the composition is applied to the skin. The phrase “cosmetically acceptable vehicle,” as used herein, means one or more compatible solid or liquid fillers, diluents, extenders and the like, which are cosmetically acceptable as defined herein. The term “compatible,” as used herein, means that the components of the compositions of this invention are capable of being commingled with the primary active of the present invention, and with each other, in a manner such that there is no interaction that would substantially reduce the efficacy of the composition under ordinary use situations. The type of vehicle utilized in the present invention depends on the type of product desired.

The vehicle of the present invention comprises one of three types of ethanol—absolute ethanol and ethanol denatured with methyl alcohol or isopropyl alcohol. By the term “effective amount”, it is meant an amount that prevents or reduces discoloration the cosmetic composition that is associated with oxidation, decomposition or degradation of hydroquinone or its derivatives. The amount of ethanol in the cosmetic compositions of the present invention generally ranges from about 1 to about 100%, preferably from about 10 to about 50%, and more preferably about 15 to about 40% by weight. In the embodiments containing ethanol denatured with methyl alcohol or isopropyl alcohol (“the ethanol composition”), the amount of the methyl alcohol or isopropyl alcohol generally ranges from about 0.0001% to about 20%, and preferably from about 0.001% to about 10% of the total amount of the ethanol composition. Absolute ethanol and ethanol denatured with methyl alcohol or isopropyl alcohol are commercially available (e.g., AAPEP Alcohol and Chemical Co. (Shelbyville, Ky.)).

The vehicle may contain at least one other element depending upon the nature of the desired product, and provided that it is compatible with the ethanol of the present invention. The vehicles may also include but are not limited to one or more of organic solvents, thickeners, humectants, oils, silicone oils, water, emulsifiers, liquid or solid emollients, propellants and powders. Such mixtures may take several forms, including but not limited to solutions, dispersions, emulsions (O/W, W/O or W/O/W) such as light creams, lotions, serums, and gels.

Additional organic solvents may include but are not limited to other lower alkanols such as propanol, isopropyl alcohol and butanol (and isomers thereof), methyl ethyl ketone, tetrahydrofuran, methyl acetate and ethyl acetate, propylene glycol, polyethylene glycol, polypropylene glycol, glycerol, 1,2,4-butanetriol, 1,2,6-hexanetriol, isopropanol, sorbitol esters, butanediol, acetone, ethylene glycol monoethyl ether, diethylene glycol monobutyl ether, diethylene glycol monoethyl ether and mixtures thereof. A preferred additional organic solvent is propylene glycol.

Gels, which are preferred compositions of the present invention, are obtained using gelling or thickening agents. Gelling or thickening agents that may be useful include but are not limited to carboxyvinyl polymers, e.g., crosslinked acrylates (e.g., Carbopol 982), hydrophobically-modified acrylates (e.g., Carbopol 1382); polyacrylamides such as, for example, the crosslinked copolymers sold under the names Sepigel 305 (CTFA name: polyacrylamide/C13-C14 isoparaffin/Laureth 7) or Simulgel 600 (CTFA name: acrylamide/sodium acryloyldimethyltaurate copolymer/isohexadecane/polysorbate 80) by SEPPIC; 2-acrylamido-2-methylpropanesulphonic acid polymers and copolymers, that are optionally crosslinked and/or neutralized, for instance the poly(2-acrylamido-2-methylpropanesulphonic acid) sold by the company Hoechst under the trade name “Hostacerin AMPS” (CTFA name: ammonium polyacryldimethyltauramide); cellulose derivatives such as hydroxyethylcellulose, sodium carboxymethylcellulose, hydroxypropyl methylcellulose, hydroxypropyl cellulose, ethyl cellulose and hydroxymethyl cellulose; polysaccharides and natural and synthetic gums, e.g., xanthan gum, sclerotium, carrageenan and pectin and mixtures thereof; modified clays (bentones); metal salts of fatty acids (aluminum stearate); ethylene/acrylate copolymers; silicas and polyethylenes. Amounts of the gelling agent or thickener generally range from about 0.0001% to about 15%. In some embodiments, the amount ranges from about 0.001% to about 1%, from about 0.01% to about 0.5%, or from about 0.5 to about 15%, all based on total weight of the cosmetic composition. A preferred gelling or thickening agent is AMPS.

Representative humectants include but are not limited to glycerin, diglycerin, triglycerin, polyglycerin, ethoxylated and propoxylated glycerols polypropylene glycol, polyethylene glycol, ethylene glycol, diethylene glycol, triethylene glycol, propylene glycol, dipropylene glycol, hexylene glycol, 1,3-butylene glycol and 1,4-butylene glycol. A preferred humectant is glycerin.

Emollients and oils include but are not limited to stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rapeseed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate; etc. Mention may also be made of volatile hydrocarbonaceous oils, such as isoparaffins such as isododecane. A preferred emollient is polyethylene glycol.

Examples of silicone oils include but are not limited to polydimethylsiloxanes, cyclomethicones, phenyltrimethicone, dimethiconol and mixtures thereof. Volatile silicone oils may also be useful. Such oils include cyclic silicones having from 3 to 8 silicon atoms, e.g., cyclic silicones having 4 to 6 carbon atoms such as, for example cyclotetradimethylsiloxane, cyclopentadimethylsiloxane, and cyclohexadimethylsiloxane, and cyclocopolymers of the dimethylsiloxane/methylalkylsiloxane type, such as SILICONE FZ 3109, sold by Union Carbide, which is a dimethylsiloxane/methyloctylsiloxane cyclocopolymer, and linear silicones having from 2 to 9 silicon atoms, for example hexamethyldisiloxane, hexylheptamethyltrisiloxane, and octylheptamethyltrisiloxane. A preferred silicone oil is cyclopentadimethylsiloxane.

Water may be present. Amount of water generally ranges from about 0.1% to about 80%, and preferably from about 5.0% to about 20%, based on total weight of the composition. Thus, compositions of the present invention may be anhydrous or substantially non-anhydrous in nature.

Emulsifiers or surfactants may be present e.g., to emulsify the water and oil phases (if both are present) and/or to provide “wash off” properties. Surfactants typically employed in the compositions of the present invention include nonionic, anionic, amphoteric, and cationic surfactants. See, e.g., Encyclopedia of Chemical Technology, KIRK-OTHMER, volume 22, pp. 333-432, 3rd edition, 1979, Wiley, for the definition of the properties and (emulsifying) functions of the surfactants, in particular pp. 347-377 of this publication regarding anionic and nonionic surfactants. Examples of surfactants useful in the compositions of the invention include but are not limited to as nonionic surfactants, fatty acids, fatty alcohols, polyethoxylated fatty alcohols or polyglycerolated fatty alcohols, such as polyethoxylated stearyl alcohols or cetylstearyl alcohols, esters of fatty acid and sucrose, and glucose alkyl esters, in particular polyoxyethylenated C₁-C₆ alkyl glucose fatty esters, and as anionic surfactants, C₁₆-C₃₀ fatty acids neutralized by amines, ammonia or the alkali metal salts thereof. Examples of amphoteric surfactants include but are not limited to betaines, sultaines, hydroxysultaines, alkyl amphodiacetates, alkyl amphodipropionates, and imidazolines, or salts thereof. Other fatty acid condensates such as those formed with amino acids, proteins, and the like are suitable as well. Specific examples include cocamphodipropionate, e.g., Miranol C2M-SF (disodium cocamphodipropionate), in its salt-free form, available from Rhone-Poulenc, and Crosultaine C-50 (cocamidopropyl hydroxysultaine), available from Croda. Examples of cationic surfactants include but are not limited to quaternary amines, amine oxides and amines, e.g., alkyl amines, alkyl imidazolines, ethoxylated amines, quaternary compounds, and quaternized esters.

In embodiments where a “wash off” effect is desired, the emulsifier (detergent active) may be selected from either soap or non-soap actives. Such actives are disclosed in standard detergent textbooks for example “Surface Active Agents,” Volume I by Schwartz and Perry, and “Surface Active Agents and Detergents,” Volume II by Schwartz, Perry and Berch. The term soap denotes salts of carboxylic fatty acids. The soap may be derived from any of the triglycerides conventionally used in soap manufacture—consequently the carboxylate anions in the soap may contain from 8 to 22 carbon atoms. For soap having 18 carbon atoms, an accompanying sodium cation will generally amount to about 8% by weight. Other cations may be employed as desired for example zinc, potassium, magnesium, alkyl ammonium and aluminium. The soap may be obtained by saponifying a fat and/or a fatty acid. The fats or oils generally used in soap manufacture may be such as tallow, tallow stearines, palm oil, palm stearines, soya bean oil, fish oil, caster oil, rice bran oil, sunflower oil, coconut oil, babassu oil, palm kernel oil, and others. In the above process the fatty acids are derived from oils/fats selected from coconut, rice bran, groundnut, tallow, palm, palm kernel, cottonseed, soya bean, castor etc. The fatty acid soaps can also be synthetically prepared (e.g., by the oxidation of petroleum or by the hydrogenation of carbon monoxide by the Fischer-Tropsch process). Resin acids, such as those present in tall oil, may be used. Naphthenic acids are also suitable. Tallow fatty acids can be derived from various animal sources and generally comprise about 1-8% myristic acid, about 21-32% palmitic acid, about 14-31% stearic acid, about 0-4% palmitoleic acid, about 36-50% oleic acid and about 0-5% linoleic acid. A typical distribution is 2.5% myristic acid, 29% palmitic acid, 23% stearic acid, 2% palmitoleic acid, 41.5% oleic acid, and 3% linoleic acid. Other similar mixtures, such as those from palm oil and those derived from various animal tallow and lard are also included. Coconut oil refers to fatty acid mixtures having an approximate carbon chain length distribution of 8% C₈, 7% C₁₀, 48% C₁₂, 17% C₁₄, 8% C₁₆, 2% C₁₈, 7% oleic and 2% linoleic acids (the first six fatty acids listed being saturated). Other sources having similar carbon chain length distributions, such as palm kernel oil and babassu kernel oil, are included within the term coconut oil.

Powders, such as chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose and ethylene glycol monostearate can be selected.

Propellants, include but are not limited to propane, butane, isobutane, dimethyl ether, carbon dioxide and nitrous oxide.

Suitable solid asymmetric particles include but are not limited to fatty acid crystals, mica, talc, clays and mixtures thereof.

The cosmetic compositions of the present invention may further comprise one or more cosmetic agents or dermatological active agents e.g., agents capable of treating or preventing any sign of aging of the skin. The active agents may be chosen, for example, from additional skin whitening agents, optical brightening agents, sunscreen agents, moisturizers, free-radical scavengers, keratolytic agents, vitamins, anti-elastase and anti-collagenase agents, peptides, fatty acid derivatives, steroids, trace elements, extracts of algae and of planktons, enzymes and coenzymes, flavonoids and ceramides, α-hydroxy acids and mixtures thereof, and enhancing agents.

Other skin whitening agents include but are not limited to tyrosinase inhibitors, botanical extracts, free radical scavengers and chelating agents. Some tyrosinase inhibitors include, but are not limited to bearberry extract, orange extract, lemon extract, cucumber extract, mercaptosuccinic acid, mercaptodextran, kojic acid, derivatives of kojic acid (e.g., kojic acid monoesters such as kojic acid monobutyrate, kojic acid monocaprate, kojic acid monopalmitate, kojic acid monostearate, kojic acid monocinnamoate and kojic acid monobenzoate; kojic acid diesters such as kojic acid dibutyrate, kojic acid dipalmitate, kojic acid distearate and kojic acid dioleate), vitamin C, derivatives of vitamin C, glutathione, cysteine and its derivatives such as N-acetyl-L-cysteine and those described in U.S. Pat. No. 5,296,500, mulberry extract and its derivatives, licorice extract and its derivatives, rosemary extract and its derivatives, and mixtures thereof.

The free radical scavengers may include, but are not limited to ascorbic acid (vitamin C) and its derivatives, vitamin E, superoxide dismutase, acerola cherry extracts, and acerola cherry fermentates. The vitamin C and its derivatives may be present in any isomeric form. For example, they can all be in cis configurations, they can all be in trans configurations, or they can be in a mixture of cis and trans configurations. Non-exclusive examples of the vitamin C derivatives are, for instance, the alkyl esters of L-ascorbic acid where the alkyl portion has from 8 to 20 carbon atoms. For example, such esters include, but are not limited to L-ascorbyl palmitate, L-ascorbyl isopalmitate, L-ascorbyl dipalmitate, L-ascorbyl isostearate, L-ascorbyl distearate, L-ascorbyl diisostearate, L-ascorbyl myristate, L-ascorbyl isomyristate, L-ascorbyl 2-ethylhexanoate, L-ascorbyl di-2-ethylhexanoate, L-ascorbyl oleate and L-ascorbyl dioleate, tetrahexyl decyl ascorbate; phosphates of L-ascorbic acid such as L-ascorbyl-2-phosphate and L-ascorbyl-3-phosphate; sulfates of L-ascorbic acid such as L-ascorbyl-2-sulfate and L-acorbyl-3-sulfate; their salts with alkaline earth metals such as calcium and magnesium. A preferred whitener is magnesium ascorbyl phosphate. The vitamin C derivatives can be used alone or in a mixture of two or more.

Other skin-whitening agents may include but are not limited to gingko extract, carob extract, rose fruit extract, geranium herb extract, Perilla extract, cinnamon extract, sweet marjoram extract, Arnica extract, Concha Blanca extract, cola ed Caballo, Piri-Piri, Pinon Negro, Pinon Blanco, extracts of clove, alfalfa, Baliospermum montanum, Melia azadirachta, Convolvulus arvensis, Gaiyo, Sansonin, Syuroyo, Seimkko, Soukyo, Taiso, Hakusempi, Woodfordia fructosa, Lagerstroemia speciosa, passiflorine, tepezcohite, amoule, Hobiyu, Baffalo uri, Achote, Guayule, Adhatoda, Cymbopogon nardus, Desmodium gangeticum, Murraya koenigii, Smilax zeylanica, Gastrodia elata, Karukeija, Biota orientalis, Kichiascoporia, Arecatachu, Phyllostachys nigra leaves, Atractylodes japonica, Koidzumi, Tila, Camotede azafran, Jamaica, Poleo verde, Navo negro, Cyperus, Kanzo, Broussonetia, Karojitsu, Trichosanthis radix, Dioscorea phizoma, and Aquilliaria.

Yet other skin-whitening agents may include but are not limited to teprenone, dihydroxy-isoquinoline, indomethacin, 3-hydroxymanule, vitamin K (such as vitamin K1-K7, its homologues, salts, and derivatives), thiazolidinone derivatives, and kynurenine and its derivatives and salts, retinol and its derivatives (e.g., Tretinoin, retinoic acid), resorcinol and its derivatives (e.g., 4-alkyl resorcinols, etc.), reservatol, placenta extracts, ellagic acid, linoleic acid and α-lipoic acid, and aminophenols e.g., such as those described in U.S. Pat. No. 6,203,781 (formula I). Amounts of additional skin whitening agents generally range from about 0.1% to about 20%, based on total weight of the composition. A preferred skin-whitening agent is α-lipoic acid.

Optical brighteners are described in “Fluorescent Whitening Agent, Encyclopedia of Chemical Technology, Kirk-Othmer,” Vol. 11, pp. 227-241, 4th edition, 1994, Wiley. Optical brighteners can be defined more particularly as compounds which absorb in the UVA range between 300 and 390 nm and re-emit essentially between 400 and 525 nm. Examples include but are not limited to stilbene derivatives (e.g., sodium 4,4′-bis[(4,6-dianilino-1,3,5-triazin-2-yl)amino]stilbene-2,2′-disulphonate), coumarin derivatives, oxazole and benzoxazole derivatives (e.g., 2,5-thiophenediylbis(5-tert-butyl-1,3-benzoxazole)) and imidalzole-derivatives. Amounts of optical brighteners generally range from about 0.1% to about 5.0%, based on total weight of the composition. A preferred brightener is oxazole.

Among the agents capable of screening out UV rays, hydrophilic or lipophilic organic screening agents, as well as inorganic sunscreen agents may be used effectively in the present invention. It is well known that chemical absorbers are classified as either UV-A or UV-B absorbers, depending on the type of radiation they protect against. UV-A absorbers generally absorb radiation in the 320 to 400 nm region of the ultraviolet spectrum. UV-A absorbers include anthranilates, benzophenones, and dibenzoyl methanes. UV-B absorbers generally absorb radiation in the 280 to 320 nm region of the ultraviolet spectrum. UV-B absorbers include p-aminobenzoic acid derivatives, cinnamates, and salicylates.

Classifying the chemical absorbers generally as UV-A or UV-B absorbers is accepted within the industry. However, a more precise classification is one based upon the chemical properties of the sunscreens. There are eight major classifications of sunscreen chemical properties that are discussed at length in “Sunscreens—Development, Evaluation and Regulatory Aspects,” by N. Shaath et al., 2nd. Edition, pages 269-273, Marcel Dekker, Inc. (1997).

The sunscreens useful in the present invention typically comprise chemical absorbers, but may also comprise physical blockers. Exemplary sunscreens which may be formulated into the compositions of the present invention are chemical absorbers such as p-aminobenzoic acid derivatives, anthranilates, benzophenones, cinnamic derivatives, dibenzoyl methanes (such as avobenzone also known as Parsol®1789), diphenylacrylate derivatives, salicylic derivatives, triazine derivatives, benzimidazole compounds, bis-benzoazolyl derivatives, methylene bis-(hydroxyphenylbenzotriazole) compounds, the sunscreen polymers and silicones, or mixtures thereof. These are variously described in U.S. Pat. Nos. 2,463,264, 4,367,390, 5,166,355 and 5,237,071 and in EP 863,145, EP 517,104, EP 570,838, EP 796,851, EP 775,698, EP 878,469, EP 933,376, EP 893,119, EP 669,323, GB 2,303,549, DE 1,972,184 and WO 93/04665. Also exemplary of the sunscreens which may be formulated into the compositions of this invention are physical blockers such as cerium oxides, chromium oxides, cobalt oxides, iron oxides, red petrolatum, silicone-treated titanium dioxide, titanium dioxide, zinc oxide, and/or zirconium oxide, or mixtures thereof.

A wide variety of sunscreens is described in U.S. Pat. Nos. 5,087,445 and 5,073,372, and Chapter VIII of Cosmetics and Science and Technology (1957) by Segarin, et al., pages 189 et seq. Sunscreens which may be formulated into the compositions of the instant invention include but are not limited to those selected from among: aminobenzoic acid, amyldimethyl PABA, cinoxate, diethanolamine p-methoxycinnamate, digalloyl trioleate, dioxybenzone, 2-ethoxyethyl p-methoxycinnamate, ethyl 4-bis(hydroxypropyl)aminobenzoate, 2-ethylhexyl-2-cyano-3,3-diphenylacrylate, ethylhexyl p-methoxycinnamate, 2-ethylhexyl salicylate, glyceryl aminobenzoate, homomenthyl salicylate, homosalate, 3-imidazol-4-ylacrylic acid and ethyl ester, methyl anthranilate, octyldimethyl PABA, red petrolatum, sulisobenzone, titanium dioxide, triethanolamine salicylate, N, N, N-trimethyl-4-(2-oxoborn-3-ylidene methyl)anillinium methyl sulfate, and mixtures thereof.

Sunscreens active in the UV-A and/or UV-B range can also include but are not limited to:
p-aminobenzoic acid,
oxyethylene (25 mol) p-aminobenzoate,
2-ethylhexyl p-dimethylaminobenzoate,
ethyl N-oxypropylene p-aminobenzoate,
glycerol p-aminobenzoate,
4-isopropylbenzyl salicylate,
2-ethylhexyl 4-methoxycinnamate,
methyl diisopropylcinnamate,
isoamyl 4-methoxycinnamate,
diethanolamine 4-methoxycinnamate,
3-(4′-trimethylammunium)-benzyliden-bornan-2-one methylsulfate,
2-hydroxy-4-methoxybenzophenone,
2,4-dihydroxybenzophenone,
2,2′,4,4′-tetrahydroxybenzophenone,
2,2′-dihydroxy-4,4′-dimethoxybenzophenone,
2-hydroxy-4-n-octoxybenzophenone,
2-hydroxy-4-methoxy-4′-methoxybenzophenone,
3-(4′-sulfo)benzyliden-bornan-2-one and soluble salts thereof,

urocanic acid,
2,4,6-tris[p-(2′-ethylhexyl-1′-oxycarbonyl)anilino]-1,3,5-triazine,
2-[(p-(tertiobutylamido)anilino]-4,6-bis-[(p-(2′-ethylhexyl-1′-oxycarbonyl)anilino]-1,3,5-triazine,
2,4-bis{[4-(2-ethyl-hexyloxy)]-2-hydroxy]-phenyl}-6-(4-methoxy-phenyl)-1,3,5-triazine (“TINOSORB S” marketed by Ciba),
the polymer of N-(2,4)-[(2-oxoborn-3-yliden)methyl]benzyl]-acrylamide,
1,4-bisbenzimidazolyl-phenylen-3,3′,5,5′-tetrasulfonic acid and salts thereof,

the benzalmalonate-substituted polyorganosiloxanes,

the benzotriazole-substituted polyorganosiloxanes (Drometrizole Trisiloxane), dispersed 2,2′-methylene-bis-[6-(2H-benzotriazol-2-yl)-4-(1,1,3,3-tetramethylbutyl)phenol] such as that marketed under the trademark MIXXIM BB/100 by Fairmount Chemical, or micronized in dispersed form thereof such as that were marketed under the trademark TINOSORB M by Ciba Specialty Chemicals Corp. (Tarrytown, N.Y.), and solubilized 2,2′-methylene-bis-[6-(2H-benzotriazol-2-yl)-4-(methyl)phenol] such as that marketed under the trademark MIXXIM BB/200 by Fairmount Chemical. Typically combinations of one of more of these sunscreens are used.

The dibenzoyl methane derivatives other than avobenzone are described, for example, in FR 2,326,405, FR 2,440,933 and EP 114,607.

Other dibenzoyl methane sunscreens other than avobenzone include (whether singly or in any combination):

2-methyldibenzoylmethane
4-methyldibenzoylmethane
4-isopropyldibenzoylmethane
4-tert-butyldibenzoylmethane
2,4-dimethyldibenzoylmethane
2,5-dimethyldibenzoylmethane
4,4′-diisopropyldibenzoylmethane
4,4′-dimethoxydibenzoylmethane
2-methyl-5-isopropyl-4′-methoxydibenzoylmethane
2-methyl-5-tert-butyl-4′-methoxydibenzoylmethane
2,4-dimethyl-4′-methoxydibenzoylmethane
2,6-dimethyl-4-tert-butyl-4′-methoxydibenzoylmethane.

Additional sunscreens that can be used are described in pages 2954-2955 of the International Cosmetic Ingredient Dictionary and Handbook (9th ed. 2002). In some embodiments, the sunscreen agent is octocrylene and/or avobenzone. Amounts of sunscreen agents in the inventive compositions generally ranges from about 0.1% to about 40% by weight, based on the total weight of the compositions.

Useful moisturizers include but are not limited to sodium lactate; polyols, and in particular glycerol, sorbitol and polyethylene glycols; mannitol; amino acids; hyaluronic acid; lanolin; urea and mixtures containing urea, such as NMF (“Natural Moisturizing Factor”); petroleum jelly; and mixtures thereof.

Useful free-radical scavengers include but are not limited to ethylenediaminetetraacetic acid and its salts such as sodium salt, guanosine, superoxydismutase, tocopherol (vitamin E) and its derivatives (acetate), ethoxyquine, lactoferrin, lactoperoxidase and nitroxide derivatives, superoxide dismutases, glutathione peroxidase, plant extracts with free-radical-scavenging activity, such as the aqueous extract of wheat germ sold by the company Silab under the name Detoxiline, and mixtures thereof.

Useful keratolytic agents include but are not limited to α-hydroxy acids, especially acids derived from fruit, for instance glycolic acid, lactic acid, malic acid, citric acid, tartaric acid and mandelic acid, derivatives thereof and mixtures thereof; β-hydroxy acids, for instance salicylic acid and its derivatives such as 5-n-octanoylsalicylic acid or 5-n-dodecanoylsalicylic acid; α-keto acids, for instance ascorbic acid or vitamin C and its derivatives such as its salts, for instance sodium ascorbate, magnesium ascorbyl phosphate or sodium ascorbyl phosphate; its esters, for instance ascorbyl acetate, ascorbyl palmitate and ascorbyl propionate, or its sugars, for instance glycosylated ascorbic acid, and mixtures thereof; β-keto acids; retinoids, for instance retinol (vitamin A) and its esters, retinal, retinoic acid and its derivatives, and also the retinoids described in documents FR-A-2,570,377, EP-A-199 636, EP-A-325-540 and EP-A-402-072; and mixtures thereof.

Useful vitamins, in addition to vitamins A, E and C indicated above, include but are not limited to vitamin B3 (or vitamin PP or niacinamide), vitamin B5 (or panthenol), vitamin D, vitamin F, derivatives, analogues and precursors of these vitamins and also those of vitamins A, E and C, for instance lycopenes or carotenes that are precursors of vitamin A, and mixtures thereof. Vitamin B3 derivatives include nicotinic acid esters, such as tocopherol nicotinate; amides derived from niacinamide by substitution of the hydrogen groups of—CONH₂; products of reaction with carboxylic acids and amino acids; esters of nicotinyl alcohol and of carboxylic acids such as acetic acid, salicylic acid, glycolic acid or palmitic acid. Mention may also be made of the following derivatives: 2-chloronicotinamide, 6-methylnicotinamide, 6-aminonicotinamide, N-methylnicotinamide, N,N-dimethylnicotinamide, N-(hydroxymethyl)nicotinamide, quinolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine. Other vitamin B3 derivatives which may also be mentioned include but are not limited to its mineral salts such as the chlorides, bromides, iodides and carbonates, and its organic salts, such as the salts obtained by reaction with carboxylic acids such as acetate, salicylate, glycolate, lactate, malate, citrate, mandelate and tartrate.

As vitamin B5, it is also possible to use panthenol or panthenyl alcohol or 2,4-dihydroxy-N(3-hydroxypropyl)-3,3-dimethylbutanamide, in its various forms: D-panthenol, DL-panthenol, and its derivatives and analogues, such as calcium pantothenate, pantethine, pantotheine, ethyl panthenyl ether, pangamic acid, pyridoxine and pantoyllactose, and natural compounds containing them such as royal jelly.

As vitamin D, mention may be made of 1, 25-dihydroxy vitamin D3 and its analogues, and also vitamin D analogues, such as those described in document WO-A-00/26167, such as, for example: 3-hydroxymethyl-5-{2-[3-(5-hydroxy-5- or 6-methylhexyl)-phenyl]-vinyl}-phenol, 3-[3-(5-hydroxy-1,5-dimethyl-hexyl)-phenoxymethyl]-5-hydroxymethyl--phenol, 6-[3-(3,4-bis-hydroxymethyl-benzyloxy)-phenyl]-2-methyl-hepta-3,5-dien-2-ol, 6-[3-(3,4-bis-hydroxymethyl-benzyloxy)-phenyl]-2-methyl-hexan-2-ol, 6-[3-(3,4-bis-hydroxymethyl-phenoxymethyl)-phenyl]-2-methyl-heptan-2-ol, 7-[3-(3,4-bis-hydroxymethyl-phenoxymethyl)-phenyl]-3-ethyl-octan-3-ol, 5-{2-[4-(5-hydroxy-5-methyl-hexyl)-phenyl]-vinyl or -ethyl}-benzene-1,3-diol, 5-{2-[3- or 4-(6-hydroxy-6-methyl-heptyl)phenyl]vinyl}-benzene-1,3--diol, 5-{2-[3- or 4-(6-hydroxy-6-methyl-heptyl)-phenyl]ethyl-benzene-1,3--diol, 2-hydroxymethyl-4-{2-[3- or 4-(5-hydroxy-5-methylhexyl)-phenyl]-vinyl-phenol, 2-hydroxymethyl-4-{2-[3- or 4-(6-hydroxy-6-methylheptyl)-phenyl]-vinyl}-phenol, 2-hydroxymethyl-4-{2-[3- or 4-(5-hydroxy-5-methylheptyl)-phenyl]-ethyl}-phenol, 2-hydroxymethyl-4-{2-[3- or 4-(6-hydroxy-6-methylheptyl)-phenyl]-ethyl}-phenol, 2-hydroxymethyl-5-(2-[4-(5-hydroxy-5-methyl-hexyl)phenyl]-vinyl-phenol, 6-[3-(3,4-bis-hydroxymethyl-benzyloxy)-phenyl-1,2-methyl-heptan-2-ol, 4-[3-(5-hydroxy-1,5-dimethyl-hexyl)-phenoxymethyl]2-hydroxymethyl-p-phenol, 6-[3- or 4-[2-(3,4-bis-hydroxymethyl-phenyl)-vinyl]phenyl}-2-methyl--hexan-2-ol, 7-{4-[2-(3,4-bis-hydroxymethyl-phenyl)-vinyl]phenyl}-2-methyl-heptan-2-ol, 5-{2-[3-(6-hydroxy-6-methyl-heptyl)-phenyl]-1-methylvinyl-benzene-1,3-diol, 5-{2-[3-(5-hydroxy-5-methyl-hexyl)-phenyl]-vinyl}benzene-1,3-diol, 5-[3-(6-hydroxy-6-methyl-heptyl)-phenoxymethyl]benzene-1,3-diol, 5-{2-[3-(7-hydroxy-7-methyl-oct-1-enyl)-phenyl]vinyl}-benzene-1,3-diol, 5-{2-[3-(7-hydroxy-7-methyl-octyl)-phenyl]-vinyllbenzene-1,3-diol, 4-{2-[3-(6-hydroxy-6-methyl-heptyl)-phenyl]-vinyl)benzene-1,2-diol, 3-{2-[3-(6-hydroxy-6-methyl-heptyl)-phenyl]-vinyl}phenol, 6-{3-[2-(3,5-bis-hydroxymethyl-phenyl)-vinyl]phenyl}-2-methyl-hexan--2-ol, 3-{2-[3-(7-hydroxy-7-methyl-octyl)-phenyl]-vinyl}phenol, 7-{3-[2-(3,5-bis-hydroxymethyl-phenyl)-vinyl]phenyl-2-methyl-heptan-2-ol, 7-{3-[2-(3,4-bis-hydroxymethyl-phenyl)-vinyl]phenyl}-2-methyl-heptan-2-ol, 7-{3-[2-(4-hydroxymethyl-phenyl)-vinyl]-phenyl}2-methyl-heptan-2-ol- , 4-{2-[3-(7-hydroxy-7-methyl-oct-1-enyl)-phenyl]vinyl}-benzene-1,2-diol, 7-[3-(3,4-bis-hydroxymethyl-phenylethynyl)-phenyl]2-methyl-heptan-2-ol, 5-{2-[3-(6-hydroxy-6-methyl-hept-1-enyl)-phenyl]vinyl}-benzene-1,3-diol, 5-{2-[3-(7-ethyl-7-hydroxy-non-1-enyl)-phenyl]vinyl)-benzene-1,3-diol, 5-{2-[3-(7-hydroxy-1-methoxy-1,7-dimethyl-octyl)phenyl]-vinyl-benzene-1,3-diol, 5-{2-[3-(6-hydroxy-1-methoxy-1,6-dimethyl-heptyl)phenyl]-vinyl}-benzene-1,3-diol, 5-{2-[3-(5-hydroxy-pentyl)-phenyl]-vinyl-benzene-1,3-diol, 5-{2-[3-(5-hydroxy-6-methyl-heptyl)-phenyl]-vinyl}benzene-1,3-diol, 5-{2-[3-(6-hydroxy-7-methyl-octyl)-phenyl]-vinyl)benzene-1,3-diol, 5-{2-[3-(5-hydroxy-6-methyl-hept-1-enyl)-phenyl]vinyl}-benzene-1,3-diol, 5-{2-[3-(6-hydroxy-7-methyl-oct-1-enyl)-phenyl]vinyl}-benzene-1,3-diol, 5-{2-[3-(1,6-dihydroxy-1,6-dimethyl-heptyl)-phenyl]vinyl}-benzene-1,3-diol, 5-(2-[3-(6-hydroxy-1,6-dimethyl-hept-1-enyl)-phenyl]vinyl}-benzene-1,3-diol.

Vitamin F is a mixture of essential fatty acids, that is to say of unsaturated acids containing at least one double bond, such as linoleic acid or 9,12-octadecadienoic acid, and its stereoisomers, linolenic acid in alpha form (9,12,15-octadecatrienoic acid) or the gamma form (6,9,12-octadecatrienoic acid) and stereoisomers thereof, arachidonic acid or 5,8,11,14-eicosatetraenoic acid and its stereoisomers.

Vitamin F or analogues thereof such as mixtures of unsaturated acids containing at least one double bond and especially mixtures of linoleic acid, of linolenic acid and of arachidonic acid, or compounds containing them and especially oils of plant origin containing them such as, for example, jojoba oil, may be used in the compositions of the present invention.

Useful anti-elastase agents include but are not limited to peptide derivatives and especially peptides from leguminous seeds such as those sold by Laboratoires Seriobiologiques de Nancy under the reference Parelastyl; the N-acylamino amide derivatives described in patent application FR-A-2,180,033, such as, for example, ethyl {2-[acetyl(3-trifluoromethylphenyl)amino]-3-methylbutyrylamino}acetate and {2-[acetyl-(3-trifluoromethylphenyl)amino]-3-methylbutyrylaminol acetic acid, and mixtures thereof. Anti-collagenase agents that may be mentioned include but are not limited to metalloprotease inhibitors, such as ethylenediamine acid (EDTA) and cysteine, and mixtures thereof.

Useful peptides include but are not limited to proteins (wheat or soybean protein), hydrolysates thereof, for instance those sold by the company Silab under the name Tensine, and mixtures thereof.

Useful fatty acid derivatives include but are not limited to polyunsaturated phospholipids including the essential fatty acid phospholipids from octopus, and mixtures thereof.

Useful steroids include but are not limited to DHEA or dehydroepiandrosterone, its biological precursors, its metabolites, and mixtures thereof. The expression “biological precursors” of DHEA especially means Δ5-pregnenolone, 17α-hydroxypregnenolone and 17α-hydroxypregnenolone sulphate. The expression “DHEA derivatives” means both its metabolic derivatives and its chemical derivatives. Metabolic derivatives that may especially be mentioned include Δ5-androstene-3,17-diol and especially 5-androstene-3β,17β-diol, Δ4-androstene-3,17-dione, 7-hydroxy DHEA (7α-hydroxy DHEA or 7β-hydroxy DHEA) and 7-keto-DHEA which is itself a metabolite of 7β-hydroxy DHEA. A preferred group is dehydroepiandrosterone, 5-pregnenolone, 17-hydroxypregnenolone, 17-hydroxypregnenolone sulphate, 5-androstene-3,17-diol, 4-androstene-3,17-dione, 7-hydroxy DHEA, 7-hydroxy DHEA, 7-keto-DHEA, and mixtures thereof.

Useful trace elements include but are not limited to copper, zinc, selenium, iron, magnesium and manganese, and mixtures thereof.

Useful extracts of algae include but are not limited to extracts of red or brown algae and, for example, the extract of brown algae from the Laminaria family, for instance the extracts from the species Laminaria digitata, and more particularly the product sold by the company CODIF under the name Phycosaccharides, which is a concentrated solution of an oligosaccharide obtained by controlled enzymatic depolymerization of membrane polysaccharides of a brown alga. It comprises a sequence of two uric acids, namely mannuronic acid and guluronic acid.

Useful extracts of planktons include but are not limited to plankton in aqueous dispersion (CTFA name: Vitreoscilla Ferment) sold under the name Mexoryl SAH by the company Chimex.Desquamating agents include hydroxy acids, more particularly α- and β-hydroxy acids, natural or synthetic retinoids, such as retinol, retinol esters, retinoic acid or retinal.

Enhancing agents may be present in the compositions of the present invention. They are believed to enhance or increase the efficacy of the hydroquinone (and one or more additional active agents that may be present) by increasing or enhancing the percutaneous absorption and/or penetration of the agent. Examples include but are not limited to the salicylic acid derivatives represented by formula (I) in U.S. Publication No. 2004/0162272. According to the '272 publication, such derivatives include those described in U.S. Pat. Nos. 6,159,479 and 5,558,871, FR 2,581,542, U.S. Pat. No. 4,767,750, EP 378,936, U.S. Pat. Nos. 5,267,407, 5,667,789, 5,580,549, and EP-A-570,230. Other salicylic acid derivatives useful herein include 5-n-octanoyl salicylic acid (capryloyl salicylic acid), 5-n-decanoyl salicylic acid, 5-n-dodecanoyl salicylic acid, 5-n-heptyloxy salicylic acid, 4-n-heptyloxy salicylic acid, and capryloyl salicylic acid (Trade name: Mexoryl SAB); see page 139 of the International Cosmetic Ingredient Dictionary, 6th Edition, Volume 1, published by the Cosmetic Toiletries, and Fragrance Association, 1995.

Suitable antioxidants include but are not limited to amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulphoximine compounds (for example buthionine-sulphoximines, homocysteine-sulphoximine buthionine sulphones, penta-, hexa- and heptathionine-sulphoximine) in very low tolerated doses (for example pmol to μmol/kg), and furthermore (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, vitamin C and derivatives (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (for example ZnO, ZnSO₄), selenium and derivatives thereof (for example selenium methionine), stilbenes and derivatives thereof (for example stilbene oxide, trans-stilbene oxide) and the derivatives of these active ingredients mentioned which are suitable according to the invention (salts, esters, ethers, sugars, nucleotides, nucleosides, peptides and lipids). The amount of the antioxidant in the compositions generally ranges from about 0.05% to about 5.0%, based on the total weight of the preparation.

Other cosmetically or dermatologically acceptable agents that may be used in the compositions of the invention include but are not limited to coloring agents (pigments, dyes, colorants e.g., iron oxides, titanium oxides and zinc oxides), preservatives, perfumes and fragrances, pulverulent agents, antiperspirants and/or odor absorbers, natural extracts, procyannidol oligomers, urea, caffeine, fillers, keratolytic agents, extracts of algae, fungi, plants, yeasts or bacteria, hydrolysed, partially hydrolysed or unhydrolysed proteins such as enzymes, antibacterial or bactericidal agents e.g., 2,4,4′-trichloro-2′-hydroxydiphenyl ether (triclosan) and 3,4,4′-trichlorocarbanilide (or triclocarban) and azelaic acid, matt-effect agents, for instance fibres, tensioning agents, and mixtures thereof. Amounts of such agents typically range from about 0.0001% to about 20% by weight of the composition.

Examples of preservatives include but are not limited to alkyl para-hydroxybenzoates, wherein the alkyl radical has from 1, 2, 3, 4, 5 or 6 carbon atoms and preferably from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Mixtures of preservatives may be used, e.g., the mixture of methyl-paraben, ethylparaben, propylparaben and butylparaben sold under the name Nipastat by Nipa, and the mixture of phenoxyethanol, methylparaben, ethylparaben, propylparaben and butylparaben sold under the name Phenonip, also by Nipa.

The compositions and methods of the present invention may be used on people in need thereof, such as the elderly e.g., to bleach dark or blotchy areas, Asians, women e.g., to treat hyperpigmentation caused by child bearing (chloasma gravidarum) and birth control pills (chloasma pillularae)(see U.S. Patent No. 4,466,955), and men and women alike having post-inflammatory hyperpigmentation following trauma (see U.S. Patent No. 4,835,191).

The composition can be packaged in a suitable container. The choice of container may depend upon the viscosity and intended use of the composition by the consumer. For example, a lotion or fluid cream can be packaged in a bottle or a roll-ball applicator, or a capsule, or a propellant-driven aerosol device or a container fitted with a pump suitable for finger operation. When the composition is a cream, it can simply be stored in a non-deformable bottle or squeeze container, such as a tube or a lidded jar.

The following examples further illustrate the present invention. They are not intended to be limiting in any way. Unless otherwise indicated, all parts are by weight.

### EXAMPLE 1

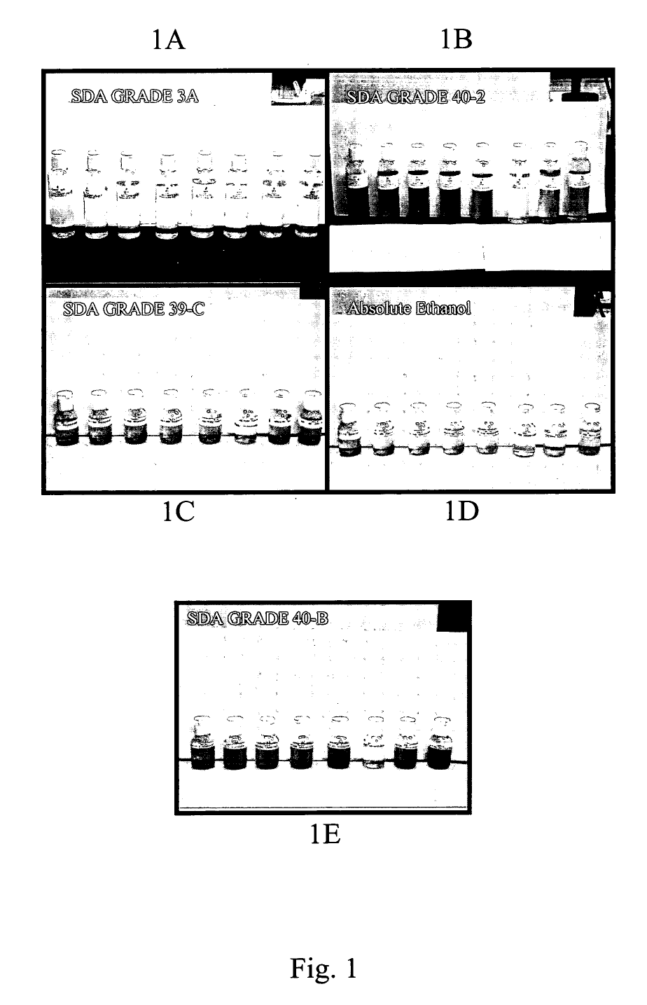

The objective of the experiments described herein was to study the effect of various denaturants on the color change of alcoholic solutions containing hydroquinone. The results of the experiments, which are also illustrated in FIGS. 1A-AE, showed that absolute ethanol and ethanol SDA grade 3A (i.e., denatured with methyl alcohol) had much lower color change as compared to ethanol SDA 40-2, 39C and 40B. Although these experiments did not involve use of ethanol denatured with isopropyl alcohol, Applicants believe that such compositions would behave similarly to those containing absolute ethanol or ethanol denatured with methyl alcohol. Unlike brucine sulfate, which is a reactive alkaloid, isopropyl alcohol is relatively non-reactive.

Solutions of hydroquinone (1% w/w) were prepared in various alcohols (i.e., grades of ethanol), and then stored at 450° C. for two weeks. Color change was monitored using a Nikon digital camera. Five different grades of ethanol, as described in Table I, were used in the experiments.

TABLE I

| Ethanol | | |
|---|---|---|
| Grade | Proof | Denaturant |
| Absolute | 200 | N/A |
| 3A | 200 | Methyl Alcohol |
| 39C | 200 | Diethyl Phthalate |
| 40-2 | 200 | Brucine Sulfate |
| 40B | 200 | Denatonium |
| | | Benzoate |

Following storage for this time period, Applicants observed that both absolute ethanol and ethanol SDA grade 3A had a much lower color change as compared to the three other grades of ethanol. These results were particularly surprising in the case of ethanol SDA 40-2 (i.e., denatured with brucine sulfate), which is a widely used grade of ethanol in the cosmetics industry.

Although these experiments did not involve use of ethanol denatured with isopropyl alcohol, Applicants believe that such compositions would behave similarly to those containing absolute ethanol or ethanol denatured with methyl alcohol. Unlike brucine sulfate, which is a reactive alkaloid, isopropyl alcohol is relatively non-reactive.

### EXAMPLE 2

### Depigmenting Formulation

| INCI Name | % | 2000 G |
|---|---|---|
| Water | 57.17 | 1143.40 |
| Hydroquinone | 2.00 | 40.00 |
| Ascorbic Acid | 0.05 | 1.00 |
| Propylene glycol | 6.00 | 120.00 |
| Tetrasodium EDTA | 0.08 | 1.60 |
| Denatured ethanol (3A) | 22.00 | 440.00 |
| Salicylic acid | 0.20 | 4.00 |
| Glycolic acid | 10.50 | 210.00 |
| AMPS | 2.00 | 40.00 |
| Total | 100.00 | 2000.00 |

EDTA, ascorbic acid, hydroquinone and glycolic acid were dissolved in water. Propylene glycol was added to the water and mixed till homogeneous. Salicylic acid was dissolved into the alcohol and then this was added to the water phase. AMPS was dispersed into the water plus alcohol phase and mixed until the polymer was hydrated completely.

### EXAMPLE 3

### Depigmenting Formulation

| INCI Name | % | 2000 G |
|---|---|---|
| Water | 62.35 | 1247.00 |
| Hydroquinone | 2.00 | 40.00 |
| Ascorbic Acid | 0.05 | 1.00 |
| Propylene glycol | 6.00 | 120.00 |
| Tetrasodium EDTA | 0.08 | 1.60 |
| Absolute ethanol (200 | 18.62 | 372.40 |
| Proof) | | |
| Salicylic acid | 0.20 | 4.00 |
| Glycolic acid | 10.50 | 210.00 |
| AMPS | .20 | 40.00 |
| Total | 100.00 | 2000.00 |

EDTA, ascorbic acid, hydroquinone and glycolic acid were dissolved in water. Propylene glycol was added to the water and mixed till homogeneous. Salicylic acid was dissolved into the alcohol and then this was added to the water phase. AMPS was dispersed into the water plus alcohol phase and mixed until the polymer was hydrated completely.

All publications cited in the specification are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore understood that numerous modifications may be made to the illustrative embodiments and that other arrangements may be devised without departing from the spirit and scope of the present invention as defined by the appended claims.

1. A composition for application to skin, comprising hydroquinone or a derivative thereof, and a cosmetically acceptable vehicle comprising an effective amount of absolute ethanol or an effective amount of an ethanol composition comprising ethanol and methyl alcohol or isopropyl alcohol as a denaturant.

2. The composition of claim 1, wherein said stabilizer comprises absolute ethanol.

3. The composition of claim 1, wherein said stabilizer comprises said ethanol composition.

4. The composition of claim 3, wherein said ethanol composition comprises methyl alcohol.

5. The composition of claim 3, wherein said ethanol composition comprises isopropyl alcohol.

6. The composition of claim 1, further comprising a sunscreen agent.

7. The composition of claim 6, wherein said sunscreen agent comprises octocrylene.

8. The composition of claim 1, wherein said vehicle further comprises a gelling agent.

9. The composition of claim 8, wherein said gelling agent comprises poly(2-acrylamido-2-methylpropanesulphonic acid).

10. The composition of claim 1, wherein said vehicle further comprises water.

11. The composition of claim 1, further comprising at least one additional skin-whitening agent other than hydroquinone.

12. The composition of claim 1, further comprising a keratolytic agent.

13. The composition of claim 12, wherein said keratolytic agent comprises an α-hydroxy acid.

14. The composition of claim 12, wherein said keratolytic agent comprises a β-hydroxy acid.

15. The composition of claim 12, wherein said keratolytic agent comprises a retinoid.

16. The composition of claim 1, further comprising an antioxidant.

17. The composition of claim 1, further comprising an emulsifier.

18. The composition of claim 1, comprising hydroquinone.

19. A method of whitening skin, comprising applying to skin a composition comprising hydroquinone or a derivative thereof, and a cosmetically acceptable vehicle comprising an effective amount of absolute ethanol or an effective amount of an ethanol composition comprising ethanol and methyl alcohol or isopropyl alcohol as a denaturant.

20. A method for reducing discoloration of compositions containing hydroquinone, comprising preparing a composition comprising hydroquinone or a derivative thereof, and a cosmetically acceptable vehicle comprising an effective amount of absolute ethanol or an effective amount of an ethanol composition comprising ethanol and methyl alcohol or isopropyl alcohol as a denaturant.
